(19) United States
(12) Patent Application Publication
(10) Pub. No.: US 2017/0080199 A1
(43) Pub. Date: Mar. 23, 2017

(54) HEMOSTATIC VALVE FOR HEART PUMP INTRODUCER

(71) Applicant: Abiomed, Inc., Danvers, MA (US)

(72) Inventor: John Murphy, North Reading, MA (US)

(21) Appl. No.: 15/267,323

(22) Filed: Sep. 16, 2016

Related U.S. Application Data
(60) Provisional application No. 62/235,440, filed on Sep. 30, 2015.
(60) Provisional application No. 62/220,684, filed on Sep. 18, 2015.

(51) Int. Cl. A61M 39/06; A61M 1/12

(52) U.S. Cl. CPC A61M 39/06; A61M 1/122; A61M 2039/0633; A61M 2039/062

(57) ABSTRACT

An introducer sheath for percutaneous insertion of a heart pump includes a tubular sheath body having a wall, a proximal end portion, a distal end portion, and an inner lumen. The introducer sheath includes a hemostatic valve disposed in the proximal end portion and forming a liquid tight seal across the inner lumen of the tubular sheath body. The hemostatic valve includes a valve cavity having a distal end and a proximal end, a first annular restriction formed within the valve cavity and having a first diameter, and a second annular restriction formed within the valve cavity distal to the first annular restriction and having a second diameter. The distal end of the valve cavity is spaced from the second annular restriction by a gap distance and a third diameter of the valve cavity along the gap distance is about equal to or greater than the first and second diameters.

### CROSS-REFERENCE TO RELATED APPLICATIONS

This application claims the benefit under 35 U.S.C. §119 (e) of U.S. Provisional Patent Application No. 62/220,684, filed Sep. 18, 2015 (pending), and U.S. Provisional Patent Application No. 62/235,440, filed Sep. 30, 2015 (pending), the contents of each of which are hereby incorporated by reference herein in their entireties.

### BACKGROUND

Patients with cardiac ailments are sometimes treated with heart pumps adapted to be inserted into the heart through adjoining blood vessels and configured to assist the natural cardiac pump function or to replace natural cardiac pump function by a continuous pumping operation.

In one common approach, an introducer sheath is used to gain vascular access prior to insertion of a heart pump. The introducer sheath includes a hemostatic valve that prevents blood leakage from the proximal end of the introducer sheath upon insertion of the introducer sheath into a blood vessel. The hemostatic valve should prevent blood leakage when guidewires or catheters required for the heart pump are inserted through the introducer sheath. The hemostatic valve should also prevent blood leakage when no objects are present in the hemostatic valve. To this end, some hemostatic valves include annular restrictions to seal around guidewires and catheters when they are inserted into the hemostatic valve, as well as features to prevent blood leakage when no objects are inserted into the hemostatic valve. The hemostatic valve should also allow retraction of objects from the hemostatic valve without requiring excessive retraction force to minimize the risk of damage to the hemostatic valve or the retracted object.

### SUMMARY

Disclosed herein is an introducer sheath featuring a hemostatic valve for percutaneous insertion of a heart pump. The hemostatic valve includes a valve cavity having annular restrictions that are sized to seal around objects of different diameters that are inserted through the hemostatic valve. The distal end of the valve cavity is spaced from the most distal annular restriction by a gap distance. The region of the valve cavity along the gap distance, or gap region, has a diameter that is about equal to or greater than the diameters of the annular restrictions. When an object is inserted into the valve cavity, the inserted object deforms portions of the hemostatic valve, especially those portions adjacent to the gap region. When an object is retracted from the valve cavity, these deformed portions flex backward into the gap region. Allowing the backward flexing into the gap region relieves stress in the sealing features, thereby reducing the risk of damage to the hemostatic valve. Thus, the gap region facilitates retraction of the object and reduces stress in the area where damage to the hemostatic valve is most likely. This gap region also reduces the overall stiffness of the hemostatic valve compared to valves lacking a gap region. The reduced stiffness can lower the insertion and retraction forces associated with the hemostatic valve.

In one aspect, an introducer sheath for percutaneous insertion of a heart pump includes a tubular sheath body having a wall, a proximal end portion, a distal end portion, and an inner lumen. The tubular sheath body also includes a hemostatic valve disposed in the proximal end portion and forming a liquid tight seal across the inner lumen of the tubular sheath body. The hemostatic valve includes a valve cavity having a distal end and a proximal end, a first annular restriction formed within the valve cavity and having a first diameter, and a second annular restriction formed within the valve cavity distal to the first annular restriction and having a second diameter. The distal end of the valve cavity is spaced from the second annular restriction by a gap distance, and a third diameter of the valve cavity along the gap distance is about equal to or greater than the first and second diameters.

In certain implementations, the diameter of the second annular restriction is about equal to or less than the diameter of the first annular restriction. In some implementations, the gap distance is about 1 mm or greater. In certain implementations, each of the first and second annular restrictions has a height along the longitudinal axis of about 0.3 mm or greater. In some implementations, the first diameter is about equal to or less than the diameter of a 9 Fr catheter. In certain implementations, the second diameter is about equal to or less than the diameter of a 6 Fr catheter. In some implementations, the force required to retract the heart pump from the hemostatic valve is about equal to or less than 20 N. In certain implementations, the force required to insert the heart pump into the hemostatic valve is about equal to or less than 20 N. In some implementations, the hemostatic valve is formed from two halves compressed together in the tubular sheath body. In certain implementations, the introducer sheath further comprises a feature disposed along a portion of the wall to facilitate tearing of the tubular sheath body and hemostatic valve along at least one predetermined path.

In another aspect, a hemostatic valve for forming a liquid tight seal across an inner lumen of a tubular sheath body includes a valve cavity having a distal end and a proximal end, a first annular restriction formed within the valve cavity and having a first diameter, and a second annular restriction formed within the valve cavity distal to the first annular restriction and having a second diameter. The distal end of the valve cavity is spaced from the second annular restriction by a gap distance, and a third diameter of the valve cavity along the gap distance is about equal to or greater than the first and second diameters.

In certain implementations, the diameter of the second annular restriction is about equal to or less than the diameter of the first annular restriction. In some implementations, the gap distance is about 1 mm or greater. In certain implementations, each of the first and second annular restrictions has a height along the longitudinal axis of about 0.3 mm or greater. In some implementations, the first diameter is about equal to or less than the diameter of a 9 Fr catheter. In certain implementations, the second diameter is about equal to or less than the diameter of a 6 Fr catheter. In some implementations, the force required to retract the heart pump from the hemostatic valve is about equal to or less than 20 N. In certain implementations, the force required to insert the heart pump into the hemostatic valve is about equal to or less than 20 N. In some implementations, the hemostatic valve is formed from two halves compressed together in the tubular sheath body.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 shows a cross-section of a hemostatic valve in the prior art;

FIG. 2 shows a cross-section of another hemostatic valve in the prior art;

FIG. 3 shows a cross-section of an illustrative hemostatic valve according to certain embodiments;

FIG. 4 shows a cross-section of an illustrative introducer sheath comprising the hemostatic valve of FIG. 3 shown in more detail;

FIG. 5 shows a cross-section of the hemostatic valve of FIG. 3 with a guidewire inserted into the valve;

FIG. 6 shows a cross-section of the hemostatic valve of FIG. 3 with a 6 F (2 mm) catheter inserted into the valve;

FIG. 7 shows a cross-section of the hemostatic valve and the 6 Fr (2 mm) catheter 602 of FIG. 6 during retraction of the catheter from the valve;

FIG. 8 shows a cross-section of the hemostatic valve of FIG. 3 with a 9 F (3 mm) catheter inserted into the valve;

FIG. 9 shows a cross-section of the hemostatic valve and the 9 Fr (3 mm) catheter of FIG. 8 during retraction of the catheter from the valve;

FIGS. 10-12 show transverse cross sections of the hemostatic valve of FIG. 3 with and without an object inserted through the valve; and

FIGS. 13-15 show a cross-section of the introducer sheath 400 of FIG. 4 before, during, and after parting.

### DETAILED DESCRIPTION

To provide an overall understanding of the systems, methods, and devices described herein, certain illustrative embodiments will be described. Although the embodiments and features described herein are specifically described for use in connection with introducer sheaths and hemostatic valves for percutaneous insertion of heart pumps, it will be understood that all the components and other features outlined below may be combined with one another in any suitable manner and may be adapted and applied to other types of introducer sheaths and hemostatic valves or other types of cardiac assist devices, including balloon pumps.

The apparatus described herein provides an introducer sheath and a hemostatic valve for percutaneous insertion of a heart pump. The hemostatic valve includes a valve cavity having annular restrictions that are sized to seal around objects of different diameters that are inserted through the hemostatic valve. The distal end of the valve cavity is spaced from the most distal annular restriction by a gap distance. The region of the valve cavity along the gap distance, or gap region, has a diameter that is about equal to or greater than the diameters of the annular restrictions. When an object is retracted from the valve, the gap region reduces the necessary retraction force and the possibility of damage to the hemostatic valve.

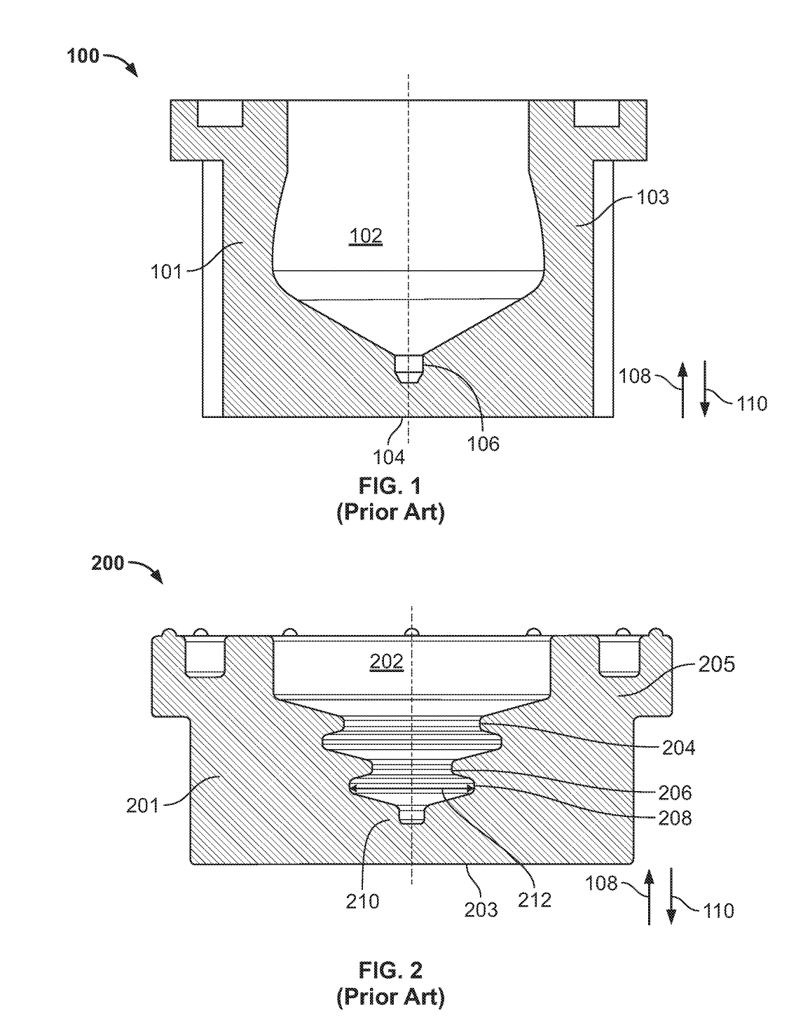

FIG. 1 shows a cross-section of a hemostatic valve in the prior art. The hemostatic valve 100 is formed from valve portion 101 and valve portion 103 compressed together. The hemostatic valve 100 includes a valve cavity 102, a valve bottom 104, and an indentation 106. The valve bottom 104 seals across the valve cavity 102 when no guidewire is inserted into hemostatic valve 100 to prevent blood from leaking through the hemostatic valve 100 in a direction 108. In addition to the valve bottom 104, the indentation 106 facilitates sealing around a guidewire inserted into the hemostatic valve 100 in a direction 110 to prevent blood from leaking through the hemostatic valve 100 in the direction 108. The hemostatic valve 100 only has two sealing features, the valve bottom 104 and the indentation 106. The valve bottom 104 deforms significantly when objects with diameters significantly larger than that of a guidewire are inserted into the hemostatic valve 100. This deformation may cause blood leakage or tearing of the hemostatic valve 100.

FIG. 2 shows a cross-section of another hemostatic valve 200 in the prior art. The hemostatic valve 200 is formed from valve portions 201 and 205 compressed together. The hemostatic valve 200 includes a valve cavity 202 and a valve bottom 203. The valve cavity 202 has a first annular restriction 204, a second annular restriction 206, an intermediate region 208, and an indentation 210. The intermediate region 208 has a diameter 212 that is smaller than the first annular restriction 204. The valve bottom 203 seals across the valve cavity 202 when no objects are inserted into hemostatic valve 200 to prevent blood from leaking in the direction 108. One or more of the first annular restriction 204, the second annular restriction 206, and the indentation 210 seal around objects inserted in the direction 110 into the hemostatic valve 200 to prevent blood from leaking in the direction 108 through the hemostatic valve 200.

When a pump (not shown) is inserted into the valve cavity 202 of the hemostatic valve 200, the pump deforms the walls of the first annular restriction 204, the second annular restriction 206, and the valve bottom 203 in the direction 110 against the intermediate region 208. This induces high stress in the walls of the valve 200 in the intermediate region 208. When the pump is retracted from the hemostatic valve 200 in the direction 108, the valve bottom flexes backward in the direction 108 into the intermediate region 208. This inversion in flexing direction is associated with high stress in the valve 200 that can cause damage (e.g., tearing) to the valve 200.

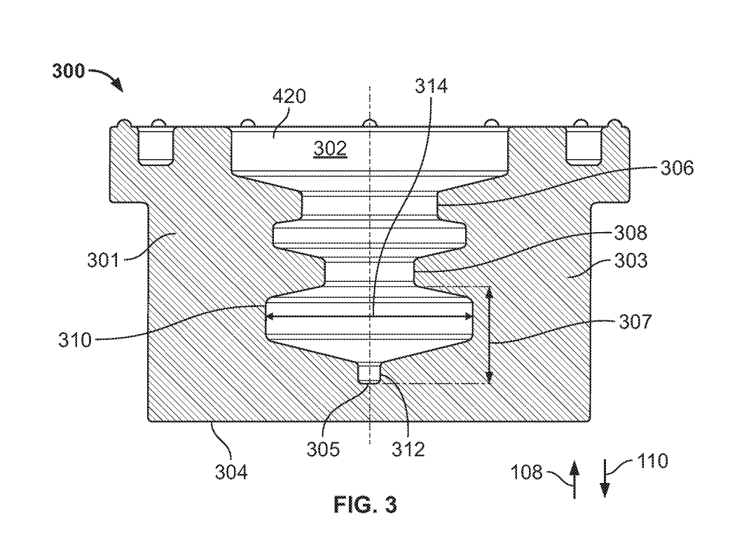

FIG. 3 shows a cross-section of an illustrative hemostatic valve 300 according to certain embodiments. The hemostatic valve 300 is formed from halves 301 and 303 compressed together. The hemostatic valve 300 includes a valve cavity 302 and a valve bottom 304. The valve cavity 302 includes a proximal end 424, a distal end 305, a first annular restriction 306, a second annular restriction 308, a gap region 310, and an indentation 312. The distal end 305 of the valve cavity 302 is spaced from the second annular restriction 308 by a gap distance 307. The gap region 310 is the region of the valve cavity 302 along the gap distance 307. The gap region 310 has a gap diameter 314 that is larger than the diameters of the first annular restriction 306 and the second annular restriction 308. In some implementations, the gap diameter 314 is about equal to the diameter of the first annular restriction 306.

The hemostatic valve 300 has four sealing features, the valve bottom 304, the first annular restriction 306, the second annular restriction 308, and the indentation 312. When no object is inserted into hemostatic valve 300, the valve bottom 304 seals across the valve cavity 302 to prevent blood from leaking in the direction 108. When an object is inserted into the hemostatic valve 300 in the direction 110, one or more of the valve bottom 304, the first annular restriction 306, the second annular restriction 308, and the indentation 312 seal around the object to prevent blood from leaking through the hemostatic valve 300 in the direction 108. The first annular restriction 306 seals around objects with diameters that are about equal to or larger than the diameter of the first annular restriction 306. The second annular restriction 308 seals around objects with diameters that are about equal to or larger than the diameter of the second annular restriction 308. The indentation 312 seals around objects with diameters that are about equal to or larger than the diameter of the indentation 312. When an object is inserted through the hemostatic valve 300 in the direction 110, the tip of the object creates an opening between the two halves of the hemostatic valve 300 at the valve bottom 304. The diameters of the first annular restriction 306, the second annular restriction 308, and the indentation 312 may be sized to seal around objects of different diameters typically inserted through the hemostatic valve 300. The objects that are inserted through the hemostatic valve 300 can include guidewires, catheters, and heart pumps. If an object with a diameter about equal to the diameter of the first annular restriction 306 is inserted into the hemostatic valve 300, the first annular restriction 306 maintains an adequate seal against blood leakage even if the inserted object excessively deforms or compromises the second annular restriction 308, the indentation 312, and the valve bottom 304.

When a pump is inserted into the hemostatic valve 300, the pump exerts friction on the walls of the valve cavity 302. This deforms the walls in the direction 110. The friction may be greatest at the valve bottom 304 because the valve 300 may be most resistant to lateral deflection at the valve bottom 300. The gap region 310 reduces the overall stiffness of the hemostatic valve 300 compared with valves lacking a gap region. The reduced stiffness of the hemostatic valve 300 can lower the force required to insert a pump into the valve. When the heart pump is retracted from the hemostatic valve 300 in the direction 108, the deformed walls of the valve bottom 304 flex backward in the direction 108 into the gap region 310. The gap region 310 relieves stress in the walls of the valve cavity 302 and the valve bottom 304 during backward flexing. Thus, the gap region 310 reduces the risk of damage to the hemostatic valve 300 during pump retraction compared to valves lacking a gap region. This also reduces the overall stiffness of the hemostatic valve 300 compared with valves lacking a gap region. The reduced stiffness of the hemostatic valve 300 can lower the force required to retract a pump through the valve and reduce the risk of damage to the hemostatic valve 300.

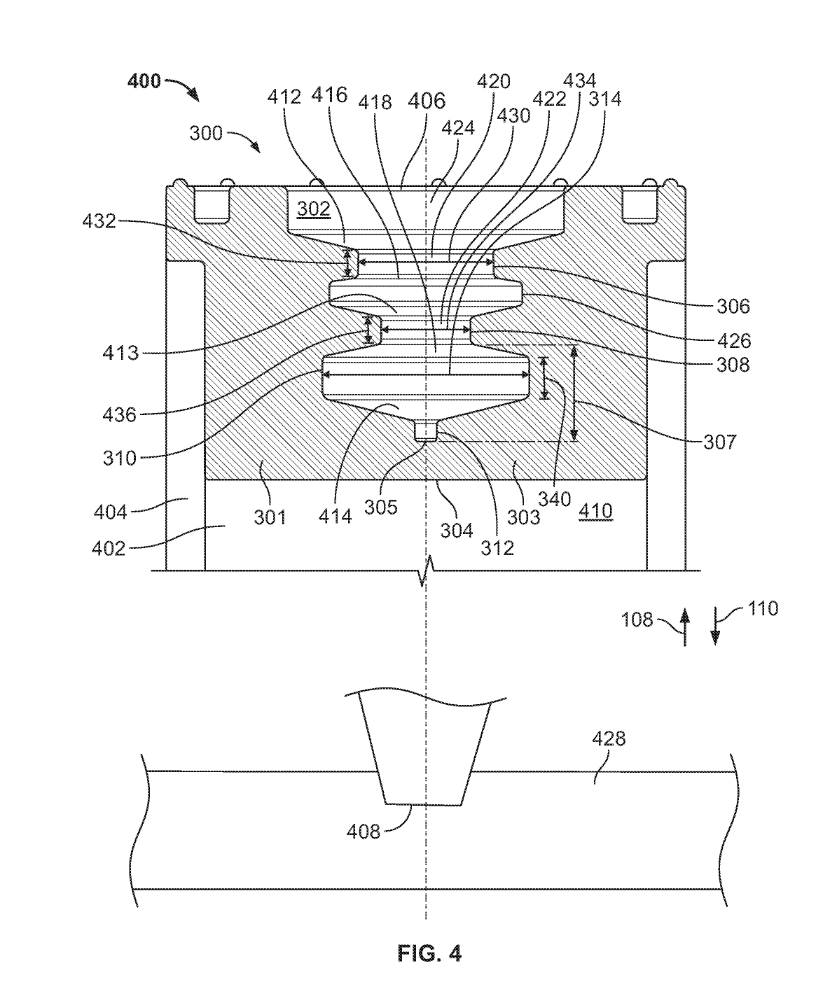

FIG. 4 shows a cross-section of an illustrative introducer sheath 400 comprising the hemostatic valve 300 of FIG. 3. The introducer sheath 400 includes a tubular sheath body 402, a wall 404, a proximal end portion 406, a distal end portion 408, an inner lumen 410, and the hemostatic valve 300. The hemostatic valve 300 includes a valve cavity 302, upper surfaces 412-414, lower surfaces 416 and 418, sealing faces 420 and 422, and a valve bottom 304. The valve cavity 302 has a proximal end 424, a first annular restriction 306, an intermediate region 426, a second annular restriction 308, a gap region 310, an indentation 312, and a distal end 305.

The introducer sheath 400 facilitates the insertion of objects, such as guidewires, catheters, and heart pumps, into a patient's vasculature. The distal end portion 408 is inserted into a patient's blood vessel 428 to create an access point into the patient's blood vessel 428 for the duration of a surgical operation. Objects are inserted into the introducer sheath 400 in the direction 110 at the proximal end portion 406 of the introducer sheath 400. The objects pass through the hemostatic valve 300 and into the introducer sheath inner lumen 410, which is defined by the wall 404. An object passing through the inner lumen 410 exits out of the distal end portion 408 and into the patient's blood vessel 428. While the introducer sheath 400 remains in place, it is possible for multiple objects to be inserted into a patient's blood vessel 428 in the direction 110. Subsequently, the objects may be retracted from the patient's blood vessel 428 in the direction 108. In certain implementations, two or more objects are inserted one after another through the access point created by the distal end portion 408. In some implementations, two or more objects are inserted simultaneously.

The hemostatic valve 300 is arranged in the inner lumen 410 of the introducer sheath 400. The hemostatic valve 300 is formed from halves 301 and 303 compressed together in the tubular sheath body 402. The proximal end 424 of the hemostatic valve 300 is located at the proximal end portion 406 of the introducer sheath 400, and the valve bottom 304 of the hemostatic valve 300 faces the distal end portion 408 of introducer sheath 400. The hemostatic valve 300 prevents blood from leaking from the patient's blood vessel 428, through the distal end portion 408 of the introducer sheath 400, and out of the proximal end portion 406 of the introducer sheath 400. The hemostatic valve 300 prevents blood leakage when no object is inserted into the introducer sheath 400. The hemostatic valve 300 also prevents blood leakage when an object is inserted into the introducer sheath 400 by sealing against the inserted object. The inserted object passes through the valve cavity 302 of the hemostatic valve 300 in the direction 110 and passes across the valve bottom 304. After the tip of the inserted object passes through the indentation 312 at the distal end 305 of the valve cavity 302, the tip of the object creates an opening between the two halves 301 and 303 of the hemostatic valve 300 at the valve bottom 304. The object then continues in the direction 110 through the inner lumen 410 of the introducer sheath 400. The object is retracted from the valve cavity 302 of the hemostatic valve 300 in the direction 108.

The first annular restriction 306 of the valve cavity 302 is defined by the sealing face 420. The first annular restriction 306 is located distal to the proximal end 424 of the valve cavity 302 and proximal to the intermediate region 426 of the valve cavity 302. The intermediate region 426 separates the first annular restriction 306 and the second annular restriction 308. The upper surface 412 of the first annular restriction 306 connects the proximal end 424 of the valve cavity 302 to the first annular restriction 306. The lower surface 416 of the first annular restriction 306 connects the first annular restriction 306 to the intermediate region 426. The first annular restriction 306 has a first diameter 430 and a first height 432. The first height 432 may be about equal to or more than 0.3 mm. For example, the first height 432 may be 0.4 mm, 0.5 mm, 0.6 mm, 0.7 mm, 0.8 mm, 0.9 mm, 1.0 mm, >1.0 mm, or any other suitable distance. The first height 432 of the first annular restriction 306 is greater than the height of the first annular restriction 204 of the prior art valve 200 in FIG. 2. This increased seal height can provide a more reliable seal against objects inserted through the first annular restriction 306. In some implementations, the first height 432 is less than 0.3 mm (e.g., 0.2 mm, 0.1 mm, <0.1 mm). The first annular restriction 306 is configured to seal around an object inserted into the hemostatic valve 300 that has a diameter about equal to the first diameter 430. When an object (e.g., a heart pump) with a diameter about equal to or greater than the first diameter 430 is inserted into the hemostatic valve 300, the first annular restriction 306 stretches and forms a seal against the object. The first diameter 430 may be about equal to or less than the diameter of a 9 Fr (3 mm) catheter. For example, the first diameter 430 may be 9 Fr (3 mm), 8 Fr (˜2.67 mm), 7 Fr (˜2.33 mm), 6 Fr (2 mm), 5 Fr (˜1.67 mm), 4 Fr (˜1.33 mm), 3 Fr (1 mm), 2 Fr (˜0.67), <1 Fr (<˜0.33 mm), or any other suitable diameter. In some implementations the first diameter 430 is greater than the diameter of a 9 Fr catheter. For example, the first diameter 430 may be 10 Fr (˜3.33 mm), 11 Fr (˜3.67 mm), 12 Fr (4 mm), 13 Fr (˜4.33 mm), 14 Fr (˜4.67 mm), or >14 Fr (>˜4.67 mm). The seal achieved in the first annular restriction 306 prevents blood from leaking from the intermediate region 426, through the proximal end 424 of the valve cavity 302, and out of the introducer sheath 400. If an object with a diameter about equal to the diameter of the first annular restriction 306 is inserted into the hemostatic valve 300, the first annular restriction 306 can maintain an adequate seal against blood leakage even if the inserted object excessively deforms the second annular restriction 308, the indentation 312, and the valve bottom 304.

The second annular restriction 308 of the valve cavity 302 is defined by the sealing face 422. The second annular restriction 308 has a second diameter 434 and a second height 436. The second diameter 434 is less than the first diameter 430. The second diameter 434 may be about equal to or less than the diameter of a 6 Fr (2 mm) catheter. For example, the second diameter 434 may be 6 Fr (2 mm), 5 Fr (˜1.67 mm), 4 Fr (˜1.33 mm), 3 Fr (1 mm), 2 Fr (˜0.67 mm), <1 Fr (<˜0.33 mm), or any other suitable diameter. In some implementations the second diameter 434 is greater than 6 Fr (2 mm). For example, the second diameter 434 may be 7 Fr (˜2.33 mm), 8 Fr (˜2.67 mm), 9 Fr (3 mm), 10 Fr (˜3.33 mm), 11 Fr (˜3.67 mm), 12 Fr (4 mm), 13 Fr (˜4.33 mm), 14 Fr (˜4.67 mm), or >14 Fr (>˜4.67 mm). The second height 436 may be about equal to or more than 0.3 mm. For example, the second height 436 may be 0.4 mm, 0.5 mm, 0.6 mm, 0.7 mm, 0.8 mm, 0.9 mm, 1.0 mm, >1.0 mm, or any other suitable distance. In some embodiments, the first diameter 430 is about equal to the second diameter 434 or less than the second diameter 434. The second height 436 of the second annular restriction 308 is greater than the height of the second annular restriction 206 of the prior art valve 200 in FIG. 2. This increased seal height can provide a more reliable seal against objects inserted through the second annular restriction 308. In some implementations, the second height 436 is less than 0.3 mm (e.g., 0.2 mm, 0.1 mm, <0.1 mm). The second annular restriction 308 is located distal to the intermediate region 426 and proximal to the gap region 310. The second annular restriction 308 is spaced from the distal end 305 of the valve cavity 302 by a gap distance 307. The upper surface 413 of the second annular restriction 308 connects the second annular restriction 308 to the intermediate region 426. The lower surface 418 of the second annular restriction 308 connects the second annular restriction 308 to the gap region 310. The second annular restriction 308 is configured to seal around an object inserted into the hemostatic valve 300 that has a diameter about equal to or slightly larger than the second diameter 434. The presence of the second annular restriction 308 in addition to the valve bottom 305 and the first annular restriction 306 improves the sealing of the valve 300 for objects having a diameter less than the first diameter 430 but larger than or equal to the second diameter 434. This is because such objects are too small to form a seal against the first annular restriction 306 and are so large that they deform the valve bottom 304 to such an extent that the valve bottom's 304 seal is compromised. Thus, the seal achieved by the second annular restriction 308 prevents blood leakage through the valve 300 when objects of an intermediate size are inserted through the valve 300.

The distal end 305 of the valve cavity 302 is spaced from the second annular restriction 308 by a gap distance 307. The gap distance 307 may be about equal to or greater than 1 mm. For example, the gap distance 307 may be 1.1 mm, 1.2 mm, 1.3 mm, 1.4 mm, 1.5 mm, 1.6 mm, 1.7 mm, 1.8 mm, 1.9 mm, 2.0 mm, >2.0 mm, or any other suitable distance. In some implementations the gap distance 307 is less than 1 mm (e.g., 0.9 mm, 0.8 mm, 0.7 mm, 0.6 mm, 0.5 mm, <0.5 mm).The gap region 310 is a portion of the valve cavity 302 along a portion of the gap distance 307. The gap region 310 connects the second annular restriction 306 to the indentation 312. The gap region 310 has a gap diameter 314. The gap diameter 314 is about equal to or larger than the first diameter 430 and the second diameter 434. When an object is inserted into the valve cavity 302, the inserted object deforms portions of the hemostatic valve 300, especially those portions adjacent to the gap region 310. When an object is retracted from the valve cavity 302, these deformed portions flex backward into the gap region 310. Allowing the backward flexing into the gap region 310 relieves stress in the sealing features, thereby reducing the risk of damage to the hemostatic valve 300. Thus, the gap region 310 facilitates retraction of the object and reduces stress in the area where damage to the hemostatic valve 300 is most likely. This gap region 310 also reduces the overall stiffness of the hemostatic valve 300 compared to valves lacking a gap region. The reduced stiffness can lower the retraction force associated with the hemostatic valve 300.

The gap region 310 has a gap height 340 that may be about equal to or greater than 0.5 mm. For example, the gap height 340 may be 0.6 mm, 0.7 mm, 0.8 mm, 0.9 mm, 1.0 mm, 1.1 mm, 1.2 mm, 1.3 mm, 1.4 mm, >1.5 mm, or any other suitable distance. In some implementations the gap height 340 is less than 0.5 mm (e.g., 0.4 mm, 0.3 mm, 0.2 mm, 0.1 mm, <0.1 mm). The gap height 340 provides a greater range of motion for the deformed walls of the hemostatic valve 300 to flex backward during retraction of an object compared with prior art devices.

The indentation 312 is formed in the valve cavity 302 distal to the gap region 310. The upper surface 414 of the valve bottom 304 connects the indentation 312 to the gap region 310. The indentation 312 seals around objects inserted into the hemostatic valve 300 that have a diameter about equal to the diameter of the indentation 312 (e.g., guidewires). The indentation 312 can also guide a guidewire through the center of the hemostatic valve 300. Centering the guidewire in this way also centers objects subsequently inserted over the guidewire.

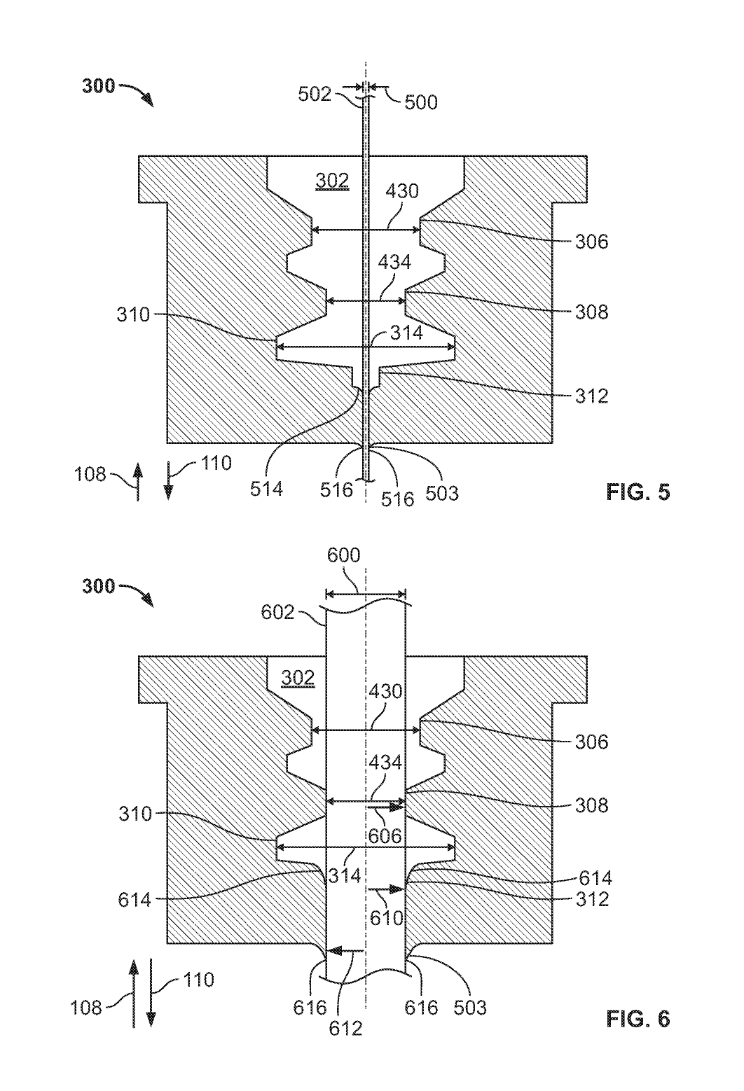

FIGS. 5-9 show cross-sections of the hemostatic valve 300 of FIG. 3 with different objects inserted through the hemostatic valve 300. It will be apparent to one skilled in the art that forces shown in FIGS. 6 and 8 are for illustrative purposes only and should not be taken to indicate that forces exist only at the location indicated. Rather, forces may exist at the location indicated and at other locations nearby. Unless otherwise indicated, all dimensions of the hemostatic valve 400 refer to the valve in the unstressed state (e.g., with no object inserted through the valve).

FIG. 5 shows a cross-section of the hemostatic valve 300 of FIG. 3 with a guidewire 502 inserted into the valve 300. The guidewire 502 has a diameter 500 that is smaller than the first diameter 430, the second diameter 434, and the gap diameter 314, but larger than the diameter of the indentation 312. For example, the diameter 500 may be 1 mm, 0.9 mm, 0.8 mm, 0.7 mm, 0.6 mm, 0.5 mm, 0.4 mm, 0.3 mm, 0.2 mm. 0.1 mm, or <0.1 mm. The guidewire 502 does not seal against the first annular restriction 306, the second annular restriction 308, or the gap region 310. However, the guidewire 502 does seal against the indentation 312 and the valve bottom 503. This seal prevents the leakage of blood in the direction 108 from the valve bottom 503 into the gap region 310. The contact between the guidewire and the walls defining the indentation 312 and the walls of the opening of the valve bottom 503 deforms the walls in the direction 110, causing deformation zones 514 and 516. Because the diameter 500 is relatively small, the deformation zones 514 and 516 are relatively small and the force required to retract the guidewire 502 is low (e.g., <20 N, <10 N, <1 N, <0.1 N).

FIG. 6 shows a cross-section of the hemostatic valve 300 of FIG. 3 with a 6 Fr (2 mm) catheter 602 inserted into the valve. The catheter 602 has a diameter 600 that is smaller than the first diameter 430 and the gap diameter 314 and about equal to the second diameter 434. The catheter 602 does not seal against the first annular restriction 306 or the gap region 310. The catheter 602 does seal against the second annular restriction 308, the indentation 312, and the valve bottom 503. This seal prevents the leakage of blood in the direction 108 from the gap region 310 into the intermediate region 426. In some implementations, the catheter 602 may deform the indentation 312 and the valve bottom 503 to such an extent that the sealing of one or more of these features may be compromised (see below description of FIGS. 10-12). In such a case, the second annular restriction 308 can still be relied on to provide a blood-tight seal across the valve 300.

The catheter 602 exerts frictional forces 606, 610, and 612 on the hemostatic valve 300 at the second annular restriction 308, the indentation 312, and the valve bottom 503, respectively. The catheter 602 contacts the walls defining the indentation 312 and the opening of the valve bottom 503, causing friction between the walls defining the indentation 312 and the opening of the valve bottom 503. The catheter 602 deforms the indentation 312 and the valve bottom 503 more than the second annular restriction 308 because the indentation 312 and the opening of the valve bottom 503 have less open volume than the second annular restriction 308. The force 610 exerted on the indentation 312 by the catheter 602 and the force 612 exerted on the valve bottom 503 by the catheter 602 are therefore larger than the force 606. The catheter 602 causes deformation zones 614 and 616 at the indentation 312 and the valve bottom 503, respectively. The gap region 310 reduces the overall stiffness of the hemostatic valve 300 compared with valves lacking a gap region. The reduced stiffness of the hemostatic valve 300 can lower the force required to insert a pump into the valve. The force required to insert the catheter 602 into the hemostatic valve 300 may be about equal to or less than 20 N. For example, the force required to insert the catheter 602 may be 20 N, 15 N, 10 N, 5 N, 1 N, or <1 N.

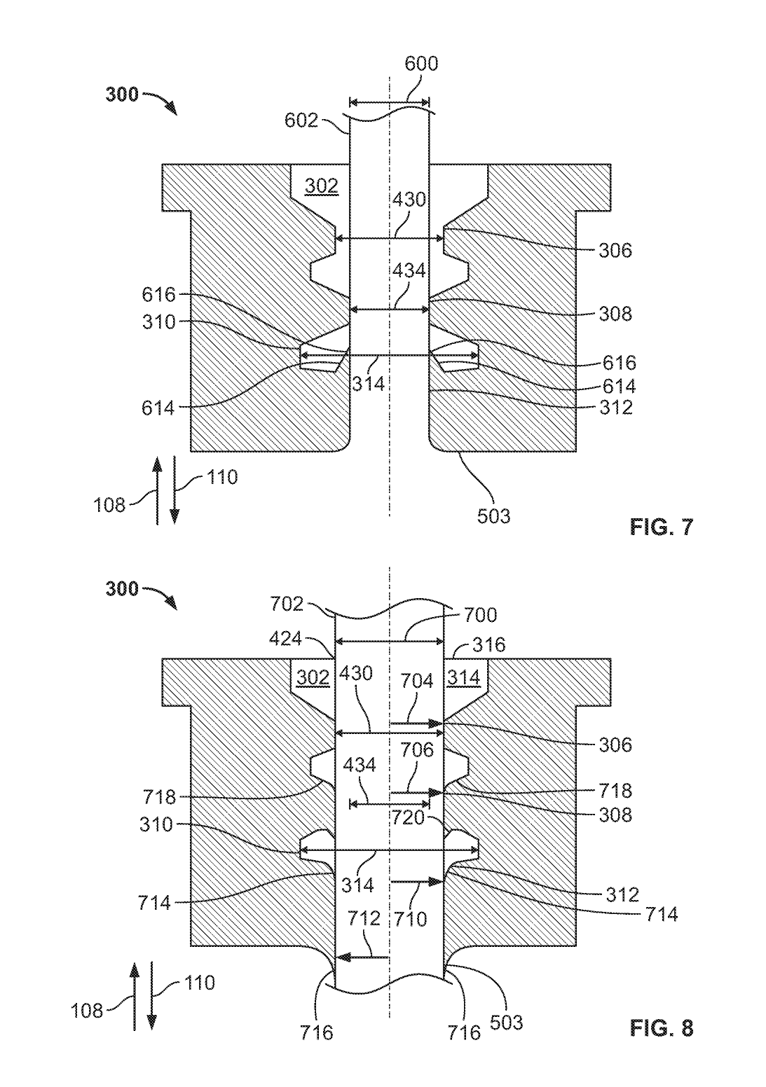

FIG. 7 shows a cross-section of the hemostatic valve 300 and the 6 Fr (2 mm) catheter 602 of FIG. 6 during retraction of the catheter 602 from the valve 300. When the catheter 602 is retracted from the hemostatic valve 300 in the direction 108, the deformation zones 614 and 616 flex backward in the direction 108 into the gap region 310. The gap region 310 relieves stress in the deformation zones 614 and 616 during backward flexing. Thus, the gap region 310 reduces the risk of damage to the hemostatic valve 300 during pump retraction compared to valves lacking a gap region. This also reduces the overall stiffness of the hemostatic valve 300 compared with valves lacking a gap region. The reduced stiffness of the hemostatic valve 300 can lower the force required to retract a pump through the valve and reduce the risk of damage to the hemostatic valve 300. The force required to retract the catheter 602 from the hemostatic valve 300 may be about equal to or less than 20 N. For example, the force required to retract the catheter 602 may be 20 N, 15 N, 10 N, 5 N, 1 N, or <1 N. During retraction, adequate sealing against blood leakage is still maintained by the second annular restriction 308.

FIG. 8 shows a cross-section of the hemostatic valve 300 of FIG. 3 with a 9 Fr (3 mm) catheter 702 inserted into the valve. The catheter 702 has a diameter 700 that is smaller than the gap diameter 314, about equal to the first diameter 430, and larger than the second diameter 434 and the diameter of the indentation 312. The catheter 702 does not seal against the gap region 310. The catheter 702 does seal against the first annular restriction 306, the second annular restriction 308, the indentation 312, and the valve bottom 503. This seal prevents leakage of blood in the direction 108 from the gap region 310 out of the proximal end 424 of the valve cavity 302. In some implementations, the catheter 702 may deform the second annular restriction 308, the indentation 312, and the valve bottom 503 to such an extent that the sealing of one or more of those features may be compromised (see below description of FIGS. 10-12). In such a case, the first annular restriction 306 can still be relied on to provide a blood-tight seal across the valve 300.

The catheter 702 exerts frictional forces 704, 706, 710, and 712 on the hemostatic valve 300 at the first annular restriction 306, the second annular restriction 308, the indentation 312, and the valve bottom 503, respectively. The catheter 702 deforms the second annular restriction 308 to a greater degree than the first annular restriction 306 because the second annular restriction 306 has a smaller diameter than the first annular restriction 306. The force 706 exerted by the catheter 702 on the second annular restriction 308 is therefore larger than the force 704. The catheter 702 causes deformation zones 718 and 720. The catheter 702 also contacts the walls defining the indentation 312 and the opening of the valve bottom 503, causing friction between the walls defining the indentation 312 and the opening of the valve bottom 503. The catheter 702 deforms the indentation 312 and the valve bottom 503 more than the second annular restriction 308 because the indentation 312 and the opening of the valve bottom 503 have less open volume than the second annular restriction 308. The force 710 exerted on the indentation 312 by the catheter 702 and the force 712 exerted on the valve bottom 503 by the catheter 702 are therefore larger than the force 706. The catheter 702 cause deformation zones 714 and 716 at the indentation 312 and the valve bottom 503, respectively. The gap region 310 reduces the overall stiffness of the hemostatic valve 300 compared with valves lacking a gap region. The reduced stiffness of the hemostatic valve 300 can lower the force required to insert a pump into the valve. The force required to insert the catheter 702 into the hemostatic valve 300 may be about equal to or less than 20 N. For example, the force required to insert the catheter 702 may be 20 N, 15 N, 10 N, 5 N, 1 N, or <1 N.

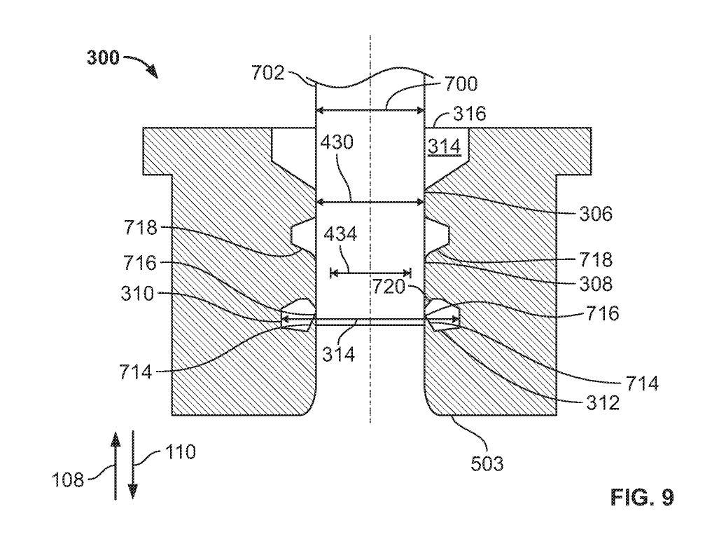

FIG. 9 shows a cross-section of the hemostatic valve 300 and the 9 Fr (3 mm) catheter 702 of FIG. 8 during retraction of the catheter 702 from the valve 300. When the catheter 702 is retracted from the hemostatic valve 300 in the direction 108, the deformation zones 714 and 716 flex backward in the direction 108 into the gap region 310. The gap region 310 relieves stress in the deformation zones 714 and 716 during backward flexing. Thus, the gap region 310 reduces the risk of damage to the hemostatic valve 300 during pump retraction compared to valves lacking a gap region. This also reduces the overall stiffness of the hemostatic valve 300 compared with valves lacking a gap region. The reduced stiffness of the hemostatic valve 300 can lower the force required to retract a pump through the valve and reduce the risk of damage to the hemostatic valve 300. The force required to retract the catheter 702 from the hemostatic valve 300 may be about equal to or less than 20 N. For example, the force required to retract the catheter 702 may be 20 N, 15 N, 10 N, 5 N, 1 N, or <1 N. During retraction, adequate sealing against blood leakage is maintained by the first annular restriction 306 and the second annular restriction 308.

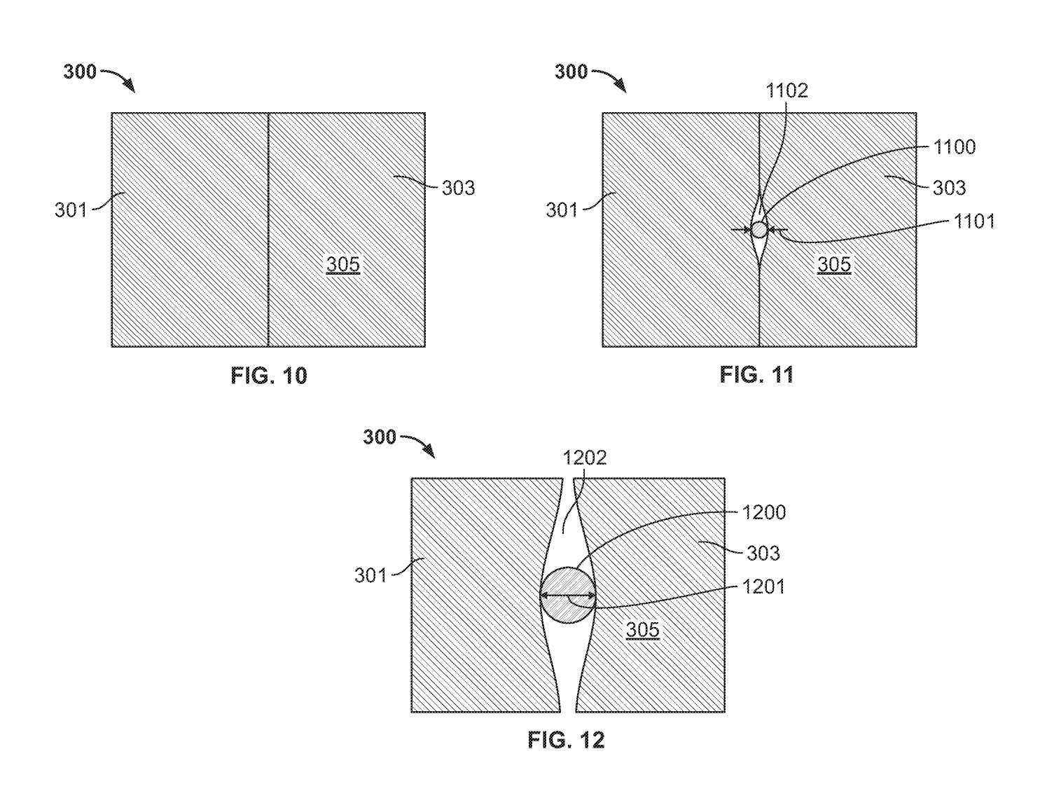

FIGS. 10-12 show transverse cross sections of the hemostatic valve of FIG. 3 with and without an object inserted through the valve. FIGS. 10-12 demonstrate how objects of different diameters deform the valve bottom 305 differently. FIG. 10 shows a transverse cross-section of the hemostatic valve 300 at the valve bottom 305. No object is inserted through the hemostatic valve 300 in FIG. 10. The hemostatic valve 300 is formed from two valve portions 301 and 303 compressed together. The valve portions 301 and 303 are compressed together such that the valve bottom 305 seals against blood leakage through the hemostatic valve 300. FIG. 11 shows a transverse cross-section of the hemostatic valve 300 with a guidewire 1100 inserted therethrough. The guidewire 1100 has a diameter 1101 and forms an opening 1102 at the valve bottom 305. The valve portion 301 and 303 remain substantially compressed together such that blood leakage through the opening 1102 is minimal. Thus, the sealing function of the valve bottom 305 is adequately maintained. FIG. 12 shows a transverse cross-section of the hemostatic valve 300 with a catheter 1200 inserted therethrough. The catheter 1200 has a diameter 1201 and forms an opening 1202 at the valve bottom 305. The diameter 1201 of the catheter 1200 is sufficiently large to cause the valve portion 301 and 303 to separate from each other at the opening 1202 such that blood leakage through the opening 1202 is substantial. Thus, the sealing function of the valve bottom 305 is compromised due to the catheter 1200. However, annular restrictions (not shown) included in the valve, such as annular restrictions 306 and 308 in FIG. 3, provide additional seals that are not compromised by insertion of the catheter 1200. Thus, the valve bottom 305 works in conjunction with the annular restrictions to form a seal around objects of various diameters.

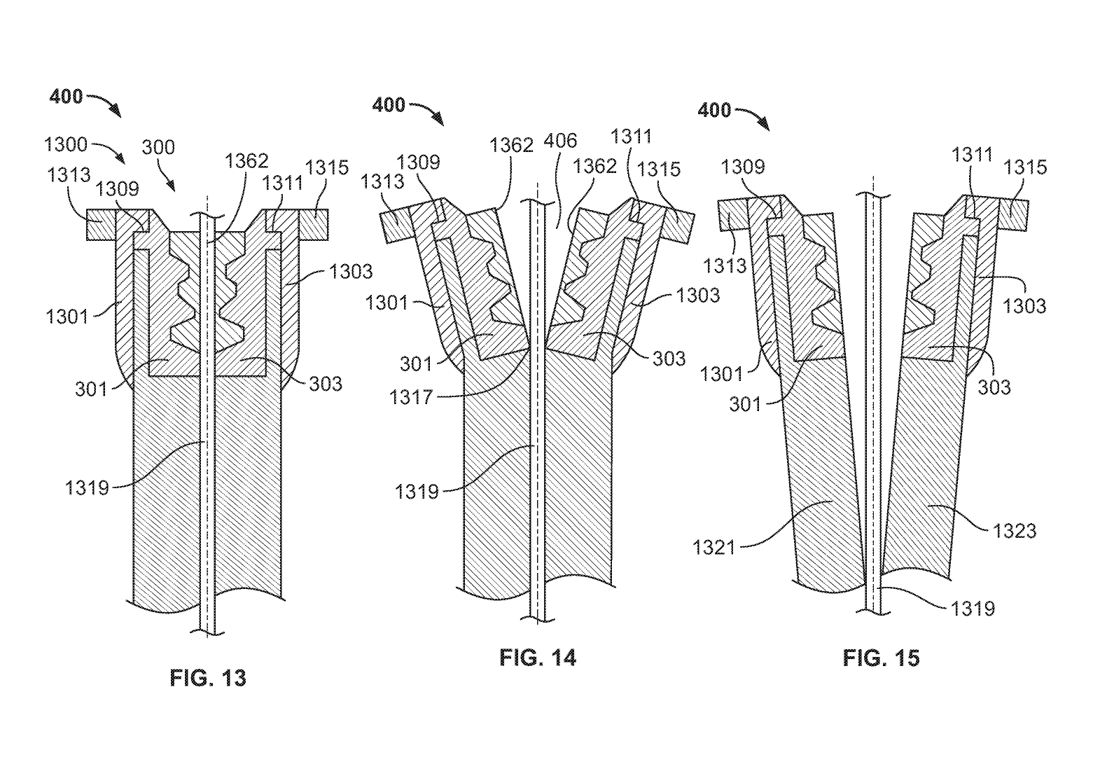

FIGS. 13-15 show a cross-section of the introducer sheath 400 of FIG. 4 before, during, and after parting. The introducer sheath 400 includes the hemostatic valve 300, a hub 1300, and a parting surface 1362. A catheter 1319 is inside the introducer sheath 400 and hemostatic valve 300. The hemostatic valve 300 is formed from valve portions 301 and 303 compressed together. The hemostatic valve 300 includes outer region portions 1309 and 1311. The valve portions 301 and 303 are held together by the hub 1300 and interface at the parting surface 1362. The parting surface 1362 separating the valve portions 301 and 303 allows the hemostatic valve 300 to be completely separated. The hub 1300 consists of hub portions 1301, 1303 and wings 1313, 1315. The valve portion 301 is connected to the hub portion 1301 at the outer region portion 1309 and the valve portion 303 is connected to the hub portion 1303 at the outer region portion 1311. The connection between the hub 1300 and the hemostatic valve 300 may be an interference fit, an adhesive bond, a connection by a mechanical fastener, or any other suitable connection. FIG. 13 shows the introducer sheath 400 before it is parted. FIG. 14 shows the introducer sheath 400 during parting. To part the introducer sheath 400 along the parting surface 1362, a healthcare professional applies force to the wings 1313 and 1315 to part (“peel-away”) the introducer sheath 400. This separates the hub into the hub portions 1301 and 1303. This also separates the hemostatic valve into the valve portions 301 and 303. This also initiates a crack 1317 in the proximal end portion 406 of the introducer sheath 400. The crack 1317 is advanced by pulling the hub portions 1301 and 1303 farther apart. This process continues until the introducer sheath 400 is completely parted into introducer sheath portions 1321 and 1323 as shown in FIG. 15. The catheter 1319 remains in place during the parting of the introducer sheath 400. It may be beneficial for the introducer sheath 400 to be removed from a patient while the catheter 1319 that was inserted through the introducer sheath 400 and into the vasculature of the patient remains in place. For example, the catheter 1319 may need to remain in the vasculature of the patient for an extended period of time while the introducer sheath 400 does not. However, it is important that removal of the introducer sheath 400 not disturb the positioning of the catheter remaining in the patient's vasculature and not prevent the placement of another sheath over the catheter. Therefore, the introducer sheath 400 is capable of being parted along the parting surface 1362 and removed while the catheter 1319 remains in place. In some embodiments, there may be more than one parting surface, and the introducer sheath 400 and the hemostatic valve 300 may be split into more than two portions.

The foregoing is merely illustrative of the principles of the disclosure, and the apparatuses can be practiced by other than the described embodiments, which are presented for purposes of illustration and not of limitation. It is to be understood that the apparatuses disclosed herein, while shown for use in percutaneous insertion of heart pumps, may be applied to apparatuses in other applications requiring hemostasis.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

We claim:

1. An introducer sheath for percutaneous insertion of a heart pump, the sheath comprising:
a tubular sheath body having a wall, a proximal end portion, a distal end portion, and an inner lumen;
a hemostatic valve disposed in the proximal end portion and forming a liquid tight seal across the inner lumen of the tubular sheath body, the hemostatic valve including:
a valve cavity having a distal end and a proximal end;
a first annular restriction formed within the valve cavity and having a first diameter;
a second annular restriction formed within the valve cavity distal to the first annular restriction and having a second diameter;
wherein the distal end of the valve cavity is spaced from the second annular restriction by a gap distance; and
wherein a third diameter of the valve cavity along the gap distance is about equal to or greater than the first and second diameters.

2. The introducer sheath of claim 1, wherein the diameter of the second annular restriction is about equal to or less than the diameter of the first annular restriction.

3. The introducer sheath of claim 1, wherein the gap distance is about 1 mm or greater.

4. The introducer sheath of claim 1, wherein each of the first and second annular restrictions has a height along the longitudinal axis of about 0.3 mm or greater.

5. The introducer sheath of claim 2, wherein the first diameter is about equal to or less than the diameter of a 9 Fr catheter.

6. The introducer sheath of claim 2, wherein the second diameter is about equal to or less than the diameter of a 6 Fr catheter.

7. The introducer sheath of claim 1, wherein the force required to retract the heart pump from the hemostatic valve is about equal to or less than 20 N.

8. The introducer sheath of claim 1, wherein the force required to insert the heart pump into the hemostatic valve is about equal to or less than 20 N.

9. The introducer sheath of claim 1, wherein the hemostatic valve is formed from two halves compressed together in the tubular sheath body.

10. The introducer sheath of claim 9, further comprising a feature disposed along a portion of the wall to facilitate tearing of the tubular sheath body and hemostatic valve along at least one predetermined path.

11. A hemostatic valve for forming a liquid tight seal across an inner lumen of a tubular sheath body, the hemostatic valve including:
a valve cavity having a distal end and a proximal end;
a first annular restriction formed within the valve cavity and having a first diameter;
a second annular restriction formed within the valve cavity distal to the first annular restriction and having a second diameter;
wherein the distal end of the valve cavity is spaced from the second annular restriction by a gap distance; and
wherein a third diameter of the valve cavity along the gap distance is about equal to or greater than the first and second diameters.

12. The hemostatic valve of claim 11, wherein the diameter of the second annular restriction is about equal to or less than the diameter of the first annular restriction.

13. The hemostatic valve of claim 11, wherein the gap distance is about 1 mm or greater.

14. The hemostatic valve of claim 11, wherein each of the first and second annular restrictions has a height along the longitudinal axis of about 0.3 mm or greater.

15. The hemostatic valve of claim 12, wherein the first diameter is about equal to or less than the diameter of a 9 Fr catheter.

16. The hemostatic valve of claim 12, wherein the second diameter is about equal to or less than the diameter of a 6 Fr catheter.

17. The hemostatic valve of claim 11, wherein the force required to retract the heart pump from the hemostatic valve is about equal to or less than 20 N.

18. The hemostatic valve of claim 11, wherein the force required to insert the heart pump into the hemostatic valve is about equal to or less than 20 N.

19. The hemostatic valve of claim 11, wherein the hemostatic valve is formed from two halves compressed together in the tubular sheath body.
